(19) United States
(12) Patent Application Publication
(10) Pub. No.: US 2021/0139599 A1
(43) Pub. Date: May 13, 2021

(54) RECOMBINANT IMMUNOGLOBULINS OF A NEW IGG5 CLASS, ENCODED BY THE HUMAN HEAVY CHAIN PSEUDO-GAMMA GENE

(71) Applicants: UNIVERSITE DE LIMOGES, Limoges Cedex (FR); CENTRE NATIONAL DE LA RECHERCHE SCIENTIFIQUE - CNRS, Paris Cedex 16 (FR); CENTRE HOSPITALIER UNIVERSITAIRE DE LIMOGES, Limoges (FR); ETABLISSEMENT FRANCAIS DU SANG, SAINT-DENIS (FR)

(72) Inventor: Michel COGNÉ, Rennes (FR)

(21) Appl. No.: 17/050,403

(22) PCT Filed: Apr. 26, 2019
(86) PCT No.: PCT/EP2019/060764

§ 371 Date: Oct. 23, 2020

(30) Foreign Application Priority Data
Apr. 26, 2018 (FR) 1853681

(51) Int. Cl. C07K 16/28

(52) U.S. Cl. CPC C07K 16/2887; C07K 2317/622; C07K 2317/52; C07K 2317/56

(57) ABSTRACT

The present invention relates to an recombinant nucleic acid molecule comprising an immunoglobulin heavy chain variable region (V_{H}), an immunoglobulin light chain variable region (V_{L}), an immunoglobulin light chain constant region (C_{L}), an immunoglobulin heavy chain constant region (C_{H}) corresponding to the CH1, hinge, CH2 and CHS protein domains encoded by the human pseudo-gamma gene (ψγ), and its uses for therapeutic or diagnostic purposes. The invention further relates to an antibody encoded by said recombinant nucleic acid molecule, and an expression cassette, vector, viral particle, host cell, transgenic organism or pharmaceutical composition comprising said recombinant nucleic acid molecule.

### FIELD OF THE INVENTION

The present invention relates to the field of medicine, in particular to a new structure of human immunoglobins encoded by a gene previously considered as a non-expressed pseudo-gene: the pseudo-gamma gene. Four immunoglobulin G classes, namely IgG1, IgG2, IgG3 and IgG4 are known ¹(Vidarsson, G., Dekkers, G. & Rispens, T. IgG subclasses and allotypes: from structure to effector functions. Front. Immunol. 5, 520 (2014)). The invention demonstrates non-conventional means for expression of the pseudo-gamma gene, and shows that products encoded by this pseudo-gene can provide a new tool for immunotherapy in human, defining an additional IgG5 class.

### BACKGROUND OF THE INVENTION

Immunotherapy is a very active field with multiple developments in terms of both cancer therapy, infectious diseases, inflammatory diseases. Antibodies targeting tumor-specific antigens or immune checkpoints have completely changed the prognosis of many tumor types, yielding long-term remission and eventually turning fatal diseases into chronic diseases almost under control by the immune system. Therapy of auto-immune disorders with monoclonal antibodies has also completely changed the prognosis of these disorders

While antibody molecules have thus became major tools against cancer and other human diseases, the diversity of molecules available is currently restricted to IgM and the four known human IgG classes (IgG1, IgG2, IgG3 and IgG4), each with unique functional properties (Vidarsson, G., Dekkers, G. & Rispens, T. IgG subclasses and allotypes: from structure to effector functions. Front. Immunol. 5, 520 (2014)). Although various mutations could be made in the structure of IgG heavy chains in order to modulate their function, the spectrum of possible changes is inherently limited by the risk of creating new immunogenic epitopes.

Only four functional genes encoding heavy chains of human IgG are present in the human genome, while a fifth homologous gene was labelled “pseudo-gamma” and explained to lack any expression due to the absence of an adequate flanking “switch” region able to support adequate recombination prior to expression (Lefranc, M. P., Lefranc, G. & Rabbitts, T. H. Inherited deletion of immunoglobulin heavy chain constant region genes in normal human individuals. Nature 300, 760-762 (1982); and Bensmana, M., Huck, S., Lefranc, G. & Lefranc, M. P. The human immunoglobulin pseudo-gamma IGHGP gene shows no major structural defect. Nucleic Acids Res. 16, 3108 (1988)).

### SUMMARY OF THE INVENTION

In a first aspect, the present invention relates to a recombinant nucleic acid molecule comprising:
a sequence encoding an immunoglobulin heavy chain variable region (V_{H});
a sequence encoding an immunoglobulin light chain variable region (V_{L});
a sequence encoding a light chain constant region (C_{L});
a sequence encoding an immunoglobulin heavy chain constant region (C_{H}) corresponding to the CH1, hinge, CH2 and CH3 protein domains encoded by the human pseudo-gamma (ψγ) gene.

Preferably, the sequence encoding an immunoglobulin heavy chain constant region encodes three or four immunoglobulin heavy chain constant domains.

The sequence encoding an immunoglobulin heavy chain constant region may encode only part of the pseudo-gamma heavy chain constant domain.

The recombinant nucleic acid molecule may further comprise additional polynucleotides for directing integration of the nucleic acid molecule by homologous recombination at a precise location into a genome of a host cell.

Also, the recombinant nucleic acid molecule may further comprise additional polynucleotides for expression as a single-chain antibody or as an hybrid antibody integrating some constant domains homologous to the classical IgG1, IgG2, IgG3 or IgG4 human IgG classes.

For instance, the combinant nucleic acid molecule may comprise peptide linkers which may fuse antibody heavy and light chains into a single-chain antibody molecule.

In another aspect, the present invention relates to an expression cassette comprising a recombinant nucleic acid molecule of the invention operably linked to one or more control sequences that direct the expression of said nucleic acid in a suitable host cell under conditions compatible with the control sequences.

In particular, the recombinant nucleic acid molecule may be operably linked to an immunoglobulin VH promoter.

In another aspect, the present invention relates to a vector comprising an expression cassette of the invention. The vector is preferably a viral vector, more preferably a retroviral vector, and even more preferably a lentiviral vector.

In preferred embodiments, said vector is an adeno-associated viral (AAV) vector, preferably AAV6 vector.

In another aspect, the present invention relates to a viral particle comprising a vector of the invention.

In a further aspect, the present invention relates to an isolated cell comprising a recombinant nucleic acid molecule, an expression cassette, a vector or a viral particle of the invention.

In preferred embodiments, the isolated cell is a mouse embryonic stem cell.

In further preferred embodiments, the isolated cell is a B cell, preferably human B cell.

In a further aspect, the present invention relates to a non-human animal transgenic organism, comprising at least one cell of the invention, preferably said transgenic organism being a mouse.

In another aspect, the present invention relates to an antibody comprising:
an immunoglobulin heavy chain variable region (VH);
an immunoglobulin light chain variable region (VL);
an immunoglobulin light chain constant region (CL);
an immunoglobulin heavy chain constant region (CH) including all or part of the peptides encoded by the human immunoglobulin pseudo-gamma gene.

The antibody of the invention may comprise:
an immunoglobulin heavy chain variable region (VH);
an immunoglobulin light chain variable region (VL);
an immunoglobulin light chain constant region (CL);
an immunoglobulin heavy chain constant region (CH) homologous to the human pseudo-gamma constant immunoglobulin gene, and sequences encoding peptide linkers wherein heavy and light chain sequences are fused into a single peptide.

The present invention also relates to a method of producing an antibody of the invention, comprising: providing a cell or a transgenic organism of the invention, said cell or organism expressing said antibody, culturing said host cell or allowing said organism to grow, and recovering said antibody from the cell culture or from a sample of said organism.

In a further aspect, the present invention also relates to a pharmaceutical composition comprising a recombinant nucleic acid molecule, an expression cassette, a vector, a viral particle a cell of the invention or an antibody of the invention, and a pharmaceutically acceptable excipient.

### BRIEF DESCRIPTION OF THE DRAWINGS

FIG. 1: A—Schematic representation of an immunoglobulin G1 class (left, prior art) and an IgG5 type of the invention (right). Compared to known IgG classes, the IgG5 constant domains are encoded by the human immunoglobulin heavy-chain pseudo-gamma gene. Structurally, among a number of differences, the hinge domain of the IgG5 differs and is slightly longer than the hinge domain of IgG1.

B—Alignment of the 5 IgG classes protein sequences (CH1, Hinge, CH2 and CH3). Grey boxes highlight differences between IgG5 and IgG1.

FIG. 2: The VDJ sequence of the rituximab was linked to the human immunoglobulin heavy-chain pseudo-gamma gene. Synthesis of this sequence was ordered from Genecust Company, and inserted in a pCDNA3.4 (+) vector (A). The rituximab V_{κ}J_{κ} and C_{κ} sequences were expressed in parallel in a second vector (B).

FIG. 3: Supernatant of transfected CHO-S cell line were analyzed by ELISA method. IgG5 molecules in supernatant were trapped by anti-Fc polyclonal antibody coated on a 96 plates. Detection was realized by addition of AP-coupled anti-IgG(H+L) polyclonal antibody. IgG1 recombinant protein was used as positive control whereas negative control was an IgM-class recombinant molecule.

FIG. 4: Flow cytometry profile: Detection of IgG5 anti-CD20 on a positive CD20 cell line (EL4-CD20), or a negative control: EL4. Target EL4 and EL4-hCD20 cell lines were incubated with purified IgG5 at 10 μg/ml during 1 hour at 2-8° C. After washes, fluorescent PE polyclonal goat anti-hIgG Fc specific antibody was incubated 30 min at 2-8° C. Cells were fixed with PFA 1% during 15 minutes and washed before flow analysis on a FORTESSA cytometer.

### DETAILED DESCRIPTION OF THE INVENTION

The inventors herein provide elements indicating that the human pseudo-gamma gene is in fact inappropriately considered as a pseudo-gene because its gene proteins were never identified at the protein level and the gene was described as lacking a flanking upstream switch region, this lack precluding normal class switch recombination and expression (Bensmana, M., Huck, S., Lefranc, G. & Lefranc, M. P. The human immunoglobulin pseudo-gamma IGHGP gene shows no major structural defect. Nucleic Acids Res. 16, 3108 (1988))³. The inventors show that DNA breaks allowing recombination upstream of the pseudo-gene can in fact be detected in human despite the lack of a switch region. They also show that functional transcripts of the pseudo-gamma gene associating a rearranged VDJ region and the pseudo-gamma gene constant sequence can also be detected by sensitive dedicated methods, demonstrating low-level expression of the pseudo-gamma gene in human. Resulting from these unexpected findings, products of the human pseudo-gamma gene will be tolerated in human and not considered as exogenous or abnormal peptides. Demonstration that such a peptide sequence can be expressed and non immunogenic in human allows to break a lock and to develop strategies for using this new class of IgG for immunotherapy in human as a secreted antibody. Indeed, the inventors also demonstrated that a recombinant immunoglobulin of this so-called IgG5 class, may be expressed as a recombinant antibody and be highly similar in structure to a classical IgG1 complete immunoglobulin. This molecule was obtained by using expression vectors encoding on one side an immunoglobulin VDJ sequence fused to the pseudo-gamma constant exons, and on the other side a light variable region fused to the C terminus of the light constant region (FIG. 1A). As shown in the experimental section of this application, the inventors demonstrated that CHO-S cells transformed with a nucleic acid encoding said immunoglobulin IgG5 could produce the recombinant antibody and that this antibody could recognize its antigen.

Accordingly, in a first aspect, the present invention relates to a recombinant nucleic acid molecule comprising, or consisting of:
a sequence encoding an immunoglobulin heavy chain variable region (V_{H});
a sequence encoding the immunoglobulin heavy chain pseudo-gamma gene constant region (ψγC_{H});
a sequence encoding an immunoglobulin light chain variable region (V_{L});
a sequence encoding a light chain constant region (CT).

As used herein, the terms “nucleic acid molecule”, “nucleic acid” and “polynucleotide” are used interchangeably and refer to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and thus can be an oligodeoxynucleoside phosphoramidate (P—NH₂) or a mixed phosphoramidate-phosphodiester oligomer. The nucleic acid of the invention can be prepared by any method known to one skilled in the art, including chemical synthesis, recombination, and mutagenesis. In preferred embodiments, the nucleic acid of the invention is a DNA molecule, preferably double stranded DNA molecule, which can be synthesized by recombinant methods well known to those skilled in the art.

A “recombinant nucleic acid” designates a nucleic acid which has been engineered and is not found as such in natural environment and in particular in wild type organisms.

The nucleic acid molecule of the invention comprises a sequence encoding an immunoglobulin heavy chain variable region (V_{H}) and a sequence encoding an immunoglobulin light chain variable region (V_{L}).

The “variable region” or “variable domain” of an antibody refers to the amino-terminal domains of the heavy or light chain of the antibody. The term “immunoglobulin heavy chain variable region” or “variable domain of the heavy chain” may be referred to as “V_{H}”. The term “immunoglobulin light chain variable region” or “variable domain of the light chain” may be referred to as “V_{L}”. These domains are generally the most variable parts of an antibody and contain the antigen-binding sites.

A light or heavy chain variable region (V_{L} or V_{H}) consists of a framework region interrupted by three hypervariable regions referred to as “complementarity determining regions” or “CDRs”. The extent of the framework region and CDRs have been precisely defined, for example as in Kabat (see “Sequences of Proteins of Immunological Interest,” E. Kabat et al., U.S. Department of Health and Human Services, (1983)). The framework region of an antibody, that is the combined framework regions of the constituent light and heavy chains, serves to position and align the CDRs, which are primarily responsible for binding to an antigen.

The nucleic acid molecule of the invention comprises a sequence encoding a light chain constant region (C_{L}).

By “constant region” as defined herein is meant an antibody-derived constant region that is encoded by one of the light or heavy chain immunoglobulin constant region genes.

By “immunoglobulin light chain constant region”, “constant light chain” or “light chain constant region” as used herein is meant the region of an antibody encoded by the kappa (Ckappa) or lambda (Clambda) light chains and may be referred to as “C_{L},”. The constant light chain typically comprises a single domain.

In some embodiments, the nucleic acid molecule of the invention could comprise, or consist of:
a sequence encoding an immunoglobulin heavy chain variable region (V_{H});
a sequence encoding an immunoglobulin light chain variable region (V_{L});
a sequence encoding a light chain constant region (C_{L});
a sequence encoding all or part of the immunoglobulin heavy chain pseudo-gamma constant region (ψγC_{H}); and
sequences encoding peptide linkers in order to express IgG5 or IgG5 fragments as a single-chain Ig.

By “immunoglobulin heavy chain constant region”, “constant heavy chain” or “heavy chain constant region” as used herein is meant the region of an antibody encoded by the mu, delta, gamma, alpha, or epsilon genes to define the antibody's isotype as IgM, IgD, IgG, IgA, or IgE, respectively. This region may be referred to as “C_{H}”.

The constant heavy chain typically comprises three or four domains. As illustration, for full length IgD, IgG or IgA antibodies, the constant heavy region, as defined herein, refers to the N-terminus of the CH1 domain to the C-terminus of the CH3 domain, and for full length IgE or IgM antibodies, the constant heavy region, as defined herein, refers to the N-terminus of the CH1 domain to the C-terminus of the CH4 domain.

Alternatively, the C_{H} region may comprise, or consist of, a fragment of the constant heavy chain of a full length antibody. For example, the C_{H} region may comprise, consist of, one or two domains such as CH1 and/or CH2.

The nucleic acid molecule of the invention comprises at least part of the pseudo-gamma gene.

In another aspect, the present invention relates to an expression cassette comprising a recombinant nucleic acid molecule of the invention operably linked to one or more control sequences that direct the expression of said nucleic acid in a suitable host cell under conditions compatible with the control sequences.

The term “control sequences” means nucleic acid sequences necessary for expression of a gene. Control sequences may be native (operably linked to the coding sequence in a naturally occurring genome) or heterologous (different from the control sequences operably linked to the coding sequence in a naturally occurring genome). Such control sequences include, but are not limited to, a leader, polyadenylation sequence, promoter, signal peptide sequence, and transcription terminator.

As used herein, the term “operably linked” refers to the association of nucleic acid sequences on a single nucleic acid molecule so that the function of one is affected by the other. For example, a promoter is operably linked with a coding sequence when it is capable of affecting the expression of that coding sequence, i.e., the coding sequence is under the transcriptional control of the promoter.

As used herein, the term “expression cassette” refers to a nucleic acid construct comprising a coding sequence and one or more control sequences required for expression of said coding sequence. Generally, the expression cassette comprises a coding sequence and regulatory sequences preceding (5′ non-coding sequences) and following (3′ non-coding sequences) the coding sequence that are required for expression of the gene product of interest. Thus, an expression cassette typically comprises a promoter sequence, a 5′ untranslated region, a coding sequence and a 3′ untranslated region that usually contains a polyadenylation site and/or transcription terminator. The expression cassette may also comprise additional regulatory elements such as, for example, enhancer sequences, a polylinker sequence facilitating the insertion of a DNA fragment within a vector and/or splicing signal sequences. The expression cassette is usually included within a vector, to facilitate cloning and transformation.

In a particular embodiment, the recombinant nucleic acid of the invention is operably linked to an immunoglobulin promoter, preferably an immunoglobulin V_{H} promoter of the contemplated host cell. Such promoter can be easily selected by the skilled person.

The expression cassette of the invention may further comprise additional sequences for directing its integration by homologous recombination at a precise location into a genome of a host cell. These sequences may be as defined above for the recombinant nucleic acid molecule.

All the embodiments of the recombinant nucleic acid molecule are also contemplated in this aspect.

In another aspect, the present invention relates a vector comprising a recombinant nucleic acid molecule or an expression cassette of the invention.

By “vector” is meant a nucleic acid molecule, preferably a DNA molecule derived, for example, from a plasmid, bacteriophage or virus, into which a nucleic acid sequence may be inserted or cloned. Non-limiting examples of vectors include plasmids, phages, cosmids, phagemids, yeast artificial chromosomes (YAC), bacterial artificial chromosomes (BAC), human artificial chromosomes (HAC), viral vectors such as adenoviral vectors or retroviral vectors, and other DNA sequences which are conventionally used in genetic engineering and/or able to convey a desired DNA sequence to a desired location within a host cell.

A vector preferably contains one or more restriction sites and may be capable of autonomous replication in a defined host cell including a target cell or tissue or a progenitor cell or tissue thereof, or be partially or entirely integrable with the genome of the defined host such that the cloned sequence is reproducible. Accordingly, the vector may be an autonomously replicating vector, i.e. a vector that exists as an extrachromosomal entity, the replication of which is independent of chromosomal replication, e.g. a linear or closed circular plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. The choice of the vector will typically depend on the compatibility of the vector with the host cell into which the vector is to be introduced.

The vector may further comprise one or more nucleic acid sequences encoding selectable marker such as auxotrophic markers (e.g., LEU2, URA3, TRP 1 or HIS3), detectable labels such as fluorescent or luminescent proteins (e.g., GFP, eGFP, DsRed, CFP), or protein conferring resistance to a chemical/toxic compound (e.g., MGMT gene conferring resistance to temozolomide). These markers can be used to select or detect host cells comprising the vector and can be easily chosen by the skilled person according to the host cell.

The vector of the invention is preferably a viral genome vector including any element required to establish the expression of the recombinant nucleic acid molecule of the invention in a host cell such as, for example, a promoter, an ITR, a ribosome binding element, terminator, enhancer, selection marker, intron, polyA signal, and/or origin of replication.

In some embodiments, the vector is a viral vector, such as vectors derived from Moloney murine leukemia virus vectors (MoMLV), MSCV, SFFV, MPSV or SNV, lentiviral vectors (e.g. derived from human immunodeficiency virus (HIV), simian immunodeficiency virus (SIV), feline immunodeficiency virus (FIV), bovine immunodeficiency virus (BIV) or equine infectious anemia virus (EIAV)), adenoviral (Ad) vectors, adeno-associated viral (AAV) vectors, simian virus 40 (SV-40) vectors, bovine papilloma virus vectors, Epstein-Barr virus, herpes virus vectors, vaccinia virus vectors, Harvey murine sarcoma virus vectors, murine mammary tumor virus vectors, Rous sarcoma virus vectors.

In particular embodiments, the vector is a retroviral vector, preferably a lentiviral vector or a non-pathogenic parvovirus.

As known in the art, depending on the specific viral vector considered for use, suitable sequences should be introduced in the vector of the invention for obtaining a functional viral vector, such as AAV ITRs for an AAV vector, or LTRs for lentiviral vectors.

The recombinant nucleic acid molecule or expression cassette of the invention may be introduced into the vector by any method known by the skilled person.

All the embodiments of the recombinant nucleic acid molecule and expression cassette of the invention are also contemplated in this aspect.

The vector of the invention may be packaged into a virus capsid to generate a “viral particle”. Thus, in a further aspect, the present invention also relates to a viral particle comprising a vector of the invention.

All the embodiments of the recombinant nucleic acid molecule, the expression cassette or the vector of the invention are also contemplated in this aspect.

In another aspect, the present invention also relates to an isolated host cell comprising, transformed or transfected, with an expression cassette, vector or viral particle of the invention.

The host cell may comprise one or several recombinant nucleic acids, expression cassettes or vectors of the invention.

The term “host cell” also encompasses any progeny of a parent host cell that is not identical to the parent host cell due to mutations that occur during replication.

The term “cell” or “host cell” includes any cell that is suitable for expressing a recombinant nucleic acid molecule of the invention. Suitable host cells for expression of antibody-encoding vectors include prokaryotic or eukaryotic cells described herein.

For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli). After expression, the antibody may be isolated from the bacterial cell lysate in a soluble fraction and can be further purified.

In addition to prokaryotes, eukaryotic microbes such as filamentous fungi or yeast are suitable expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22: 1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See, e.g., U.S. Pat. Nos. 5,959,177, 6,040,498, 6,420,548, 7,125,978, and 6,417,429.

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse Sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MT 060562); TRI cells, as described, e.g., in Mather et al., Annals N. Y. Acad. Sci. 383:44-68 (1982); MRC 5 cells; and FS4 cells. Other useful mammalian host cell lines include Chinese hamster ovary (CHO) cells, including DHFR CHO cells (Urlaub et al., Proc. Natl. Acad. Sci. USA 77:4216 (1980)); and myeloma cell lines such as YO, NSO and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

In a particular embodiment, the host cell is selected from mycobacteria cells, fungal cells, yeast cells, plant cells, insect cells, non-human animal cells, human cells, or cell fusions such as, for example, hybridomas. Preferably, the cell is selected from human, primate, rabbit or rodents (e.g., mice, rats, hamsters, guinea pigs) cells. More preferably, the cell is selected from human and mouse cells. Even more preferably, the cell is a human cell.

In preferred embodiments, the cell is a B cell, preferably a human or mouse B cell, more preferably a human B cell. The host cell, preferably the non-human cell, may also be a totipotent, pluripotent, or adult stem cell, a zygote, or a somatic cell. In an embodiment, the host cell is an embryonic stem cell, preferably a non-human embryonic stem cell, more preferably a mouse embryonic stem cell.

The expression cassette or vector of the invention may be transferred into host cells using any known technique including, but being not limited to, calcium phosphate-DNA precipitation, DEAE-Dextran transfection, electroporation, microinjection, biolistic, lipofection, or viral infection, and may be maintained in the host cell in an ectopic form or may be integrated into the genome.

In preferred embodiments, the expression cassette or vector of the invention is transferred into the host cell by viral infection, preferably using a viral particle of the invention, more preferably using an AAV particle of the invention.

All the embodiments of the recombinant nucleic acid molecule, the expression cassette, the vector or the viral particle of the invention are also contemplated in this aspect.

In another aspect, the present invention further relates to a transgenic organism, preferably a non-human transgenic organism, comprising at least one host cell of the invention. The invention also relates to a method of generating a transgenic organism comprising at least one transgenic host cell of the invention.

All embodiments of the recombinant nucleic acid molecule, the expression cassette, the vector, the viral particle and the host cell of the invention are also contemplated in this aspect.

In particular, the organism may be a non-human animal, such as primates (e.g., non-human primates such as monkeys), rabbits, or rodents (e.g., mice, rats, hamsters, guinea pigs). Preferably, the transgenic organism is a non-human mammal. More preferably, the transgenic organism is a mouse.

Methods of generating transgenic organisms, in particular transgenic mice are well-known by the skilled person. It should be understood that any of these methods can be used to practice the invention and that the methods disclosed herein are non-limitative.

In particular, the method of generating a transgenic organism may comprise:
introducing an expression cassette or a vector of the invention in a non-human embryonic stem cell;
obtaining a transgenic embryonic stem cell wherein the recombinant nucleic acid molecule of the invention is inserted into the genome, preferably by homologous recombination;
injecting said transgenic embryonic stem cell into a blastocyst of a non-human animal to form chimeras; and
reimplanting said injected blastocyst into a foster mother.

Embryonic stem (ES) cell are typically obtained from pre-implantation embryos cultured in vitro. Preferably, the cassette or vector of the invention is transfected into said ES cell by electroporation. The ES cells are cultured and prepared for transfection using methods known in the related art. The ES cells that will be transfected with the cassette or vector of the invention are derived from embryo or blastocyst of the same species as the developing embryo or blastocyst into which they are to be introduced. ES cells are typically selected for their ability to integrate into the inner cell mass and contribute to the germ line of an individual when introduced into the animal in an embryo at the blastocyst stage of development. In one embodiment, the ES cells are isolated from the mouse blastocysts.

After transfection into the ES cells, the recombinant nucleic acid molecule of the invention integrates with the genomic DNA of the cell in order to produce an antibody of the invention as defined below.

After transfection, the ES cells are cultured under suitable condition to detect transfected cells. For example, when the cassette or vector comprises a marker gene, e.g. an antibiotic resistant marker, e.g. neomycin resistant gene, the cells are cultured in that antibiotic. The DNA and/or protein expression of the surviving ES cells may be analyzed using Southern Blot technology in order to verify the proper integration of the cassette.

The selected ES cells are then injected into a blastocyst of a non-human animal to form chimeras. The non-human animal is preferably a mouse, a hamster, a rat or a rabbit. More preferably, the non-human animal is a mouse.

In particular, the ES cells may be inserted into an early embryo using microinjection. The injected blastocysts are re-implanted into a foster mother. When the progenies are born, they are screened for the presence of the recombinant nucleic acid molecule, expression cassette or vector of the invention, e.g. using Southern Blot and/or PCR technique. The heterozygotes are identified and are then crossed with each other to generate homozygous animals.

In another embodiment, the method of generating a transgenic organism may comprise:
introducing in a non-human fertilized egg (i) an expression cassette or vector of the invention and (ii) a nuclease system used to target the cassette or vector at the correct locus by homologous recombination;
obtaining a transgenic fertilized egg wherein the expression cassette or vector of the invention is inserted into the genome by homologous recombination; and
reimplanting said injected fertilized egg into a foster mother.

The nuclease system used to target the cassette or vector at the correct locus may be any suitable system known by the skilled person, such as systems involving ZFN, TALE or CRISPR/Cas9 nucleases.

Preferably, the nuclease system is a CRISPR/Cas9 system. To use Cas9 to modify genomic sequences, the protein can be delivered directly to a cell. Alternatively, an mRNA that encodes Cas9 can be delivered to a cell, or a gene that provides for expression of an mRNA that encodes Cas9 can be delivered to a cell. In addition, either target specific crRNA and a tracrRNA or target specific gRNA(s) can be delivered to the cell (these RNAs can alternatively be produced by a gene constructed to express these RNAs). Selection of target sites and designed of crRNA/gRNA are well known in the art.

The present invention also provides cells or tissues, including immortalized cell lines and primary cells or tissues, derived from the transgenic non-human animal of the invention and its progeny.

In another aspect, the present invention relates to an antibody comprising:
an immunoglobulin heavy chain variable region (V_{H});
an immunoglobulin light chain variable region (V_{L});
an immunoglobulin light chain constant region (C_{L});
part or all of the immunoglobulin heavy chain constant region (C_{H}) from the pseudo-gamma gene.

In particular, the antibody of the invention may be any antibody obtained via the expression of the coding sequences of the recombinant nucleic acid molecule of the invention.

All embodiments of the recombinant nucleic acid molecule, the expression cassette, the vector, the viral particle, the host cell and the transgenic animal of the invention are also contemplated in this aspect.

The term “antibody” herein is used in the broadest sense and specifically covers monoclonal antibodies (including full length monoclonal antibodies), polyclonal antibodies, multispecific antibodies (e.g., bispecific antibodies), antibody fragments, and derivatives thereof, so long as they comprise a chain comprising V_{H}, V_{L}, C_{L}, pseudo-gamma C_{H}.

The antibody of the invention may be a single chain antibody comprising only one chain comprising V_{H}, V_{L}, C_{L}, part or all of pseudo-gamma C_{H}. Alternatively, the antibody of the invention may comprise at least two chains comprising V_{H}, V_{L}, C_{L}, part or all of pseudo-gamma C_{H} and as defined above, part of classical Ig, thus creating an hybrid IgG molecule.

In some embodiments, the antibody may be part of a larger fusion molecule, formed by covalent or non-covalent association of the antibody with one or more other proteins or peptides. In particular, the antibody of the invention may further comprise additional antibody domains, e.g. additional heavy and light chain domains.

In some embodiments, the antibody is a full length antibody. The term “full length antibody”, as used herein, refers to an antibody having a structure substantially similar to a native antibody structure, not antibody fragments as defined below. The terms particularly refer to an antibody with heavy chains that contain an Fc region. In preferred embodiments, the antibody in a full length IgG5 antibody.

In some other embodiments, the antibody is an antibody fragment. As used herein, the term “antibody fragment” refers to a portion of a full length antibody, preferably fragment comprising the variable domain of the heavy chain, the light chain, and at least a fragment of the N-terminus of the constant pseudo-gamma heavy chain. Antibody fragments are preferably selected from the group consisting of Fab, Fab′, F(ab)₂, F(ab′)₂ and F(ab)₃.

Antibody fragments can be made by various techniques, including but not limited to proteolytic digestion of intact antibody, well-known by the skilled person, as well as recombinant techniques described herein. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen.

By “Fab”, “Fab fragment” or “Fab region” as used herein is meant the polypeptide that comprises the V_{H}, CH1, V_{L}, and C_{L} immunoglobulin domains. Fab may refer to this region in isolation or this region in the context of a polypeptide as described herein.

Fab′ fragments differ from Fab fragments by the addition of a few residues at the carboxy terminus of the heavy chain CH1 domain including one or more cysteines from the antibody hinge region. F(ab′)2 antibody fragments originally were produced as pairs of Fab′ fragments which have hinge cysteines between them. Other chemical couplings of antibody fragments are also known.

The term “antibody derivative”, as used herein, refers to an antibody provided herein, e.g. a full-length antibody or a fragment of an antibody, wherein one or more of the amino acids are chemically modified, e.g. by alkylation, PEGylation, acylation, ester or amide formation or the like. In particular, this term may refer to an antibody provided herein that is further modified to contain additional nonproteinaceous moieties that are known in the art and readily available. The moieties suitable for derivatization of the antibody include but are not limited to water soluble polymers. Examples of water soluble polymers include, but are not limited to, PEG, copolymers of ethylene glycol/propylene glycol, carboxymethylcellulose, dextran and polyvinyl alcohol.

The derivative may also be an immunoconjugate comprising an antibody of the invention conjugated to one or more heterologous molecule(s), including but not limited to a cytotoxic agent, a detectable moiety such as a fluorescent moiety, a diagnostic radioisotope or an imaging agent; or to a solid support, such as agarose beads or the like. Examples of cytotoxic agents include, but are not limited to chemotherapeutic agents or drugs, growth inhibitory agents, toxins (e.g., protein toxins, enzymatically active toxins of bacterial, fungal, plant, or animal origin, or fragments thereof), or radioactive isotopes. Conjugates of an antibody and cytotoxic agent may be made using a variety of bifunctional protein coupling agents well known by the skilled person. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52: 127-131 (1992)) may be used.

The antibody of the invention may comprise a functional Fc region, a native sequence Fc region or a variant Fc region. A “functional Fc region” possesses an effector function of a native sequence Fc region. Exemplary “effector functions” include C1q binding; Cell Dependent Cytotoxicity (CDC); Fc receptor binding; Antibody Dependent Cell Cytotoxicity (ADCC), phagocytosis, Antibody Dependent Cell Phagocytosis (ADCP), down regulation of cell surface receptors, etc. Such effector functions generally require the Fc region to be combined with a binding domain (e.g., an antibody variable domain) and can be assessed using various well known assays.

A “native sequence Fc region” comprises an amino acid sequence identical to the amino acid sequence of an Fc region found in nature, preferably a native sequence human Fc region.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g., from about one to about ten amino acid substitutions, and preferably from about one to about five amino acid substitutions in a native sequence Fc region or in the Fc region of the parent polypeptide. The variant Fc region herein will preferably possess at least about 80% sequence identity with a native sequence Fc region and/or with an Fc region of a parent polypeptide, and most preferably at least about 90% sequence identity therewith, more preferably at least about 95% sequence identity therewith.

In certain embodiments, the antibody provided herein is a multispecific antibody, e.g. a bispecific antibody. Multispecific antibodies are monoclonal antibodies that have binding specificities for at least two different sites.

Multispecific antibodies of the invention may be obtained through the recombinant co-expression in a host cell of two recombinant nucleic acid molecule of the invention leading to two single chain antibodies of the invention. These chains naturally associate together via disulfure bridges to form a multispecific antibody comprising at least two light chains and at least two heavy chains, the variable domains of said chains recognizing at least two different epitopes.

In some embodiments, the antibody is a purified antibody. A “purified” antibody is one which has been separated from a component of its production environment, preferably separated from its producing cell and/or from other antibodies. In particular, the antibody may be purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g. Flatman et al., J. Chromatogr. B 848:79-87 (2007). The antibody may, for example, be purified from the culture medium comprising the host cell expressing a recombinant nucleic acid molecule of the invention.

Depending on the method of producing the antibody of the invention or the application, the antibody of the invention may be a polyclonal or monoclonal antibody. Preferably, the antibody is a monoclonal antibody. The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The monoclonal antibody may be made by any method known by the skilled person.

The antibody of the invention may be a chimeric, humanized or human antibody.

“Chimeric” antibodies are antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (Morrison et al., Proc. Natl. Acad Sci. USA 81:6851-6855 (1984)). Preferably, at least a portion of the framework of the antibody is a human consensus framework sequence.

“Humanized” forms of non-human (e.g., murine) antibodies are chimeric antibodies which contain minimal sequence derived from non-human immunoglobulin. For the most part, humanized antibodies are human immunoglobulins (recipient antibody) in which hypervariable region residues of the recipient are replaced by hypervariable region residues from a non-human species (donor antibody) such as mouse, rat, rabbit or non-human primate having the desired specificity, affinity, and capacity. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FRs are those of a human immunoglobulin sequence. The humanized antibody optionally also comprises at least a portion of an immunoglobulin constant region (Fc), typically that of a human immunoglobulin (Jones et al., Nature 321:522-525 (1986); Reichmann et al., Nature 332:323.329 (1988); and Presta, Curr. Op. Struct. Biol. 2:593-596 (1992)).

A “human antibody” or “fully human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues.

In preferred embodiments, the antibody of the invention is a monoclonal antibody, preferably a human monoclonal antibody.

The antibody of the invention is preferably a therapeutic antibody. The therapeutic antibody may be useful for the treatment of any disease, such as cancer, inflammatory diseases, infectious diseases or auto-immune diseases.

In particular, the antibody of the invention may comprise the variable and/or constant regions of a therapeutic antibody, preferably of an approved (e.g. EMA or FDA approved) therapeutic antibody. In a particular embodiment, the antibody of the invention comprises the variable and constant regions of a therapeutic antibody, preferably of an approved therapeutic antibody. Examples of such therapeutic antibodies include, but are not limited to, abciximab, adalimumab, alemtuzumab, alirocumab, atezolizumab, avelumab, basiliximab, belimumab, bevacizumab, bezlotoxumab, blinatumomab, brentuximab, brodalumab, canakinumab, capromab, cetuximab, daclizumab, daratumumab, denosumab, dinutuximab, dupilumab, durvalumab, eculizumab, elotuzumab, evolocumab, golimumab, ibritumomab, idarucizumab, infliximab, ipilimumab, ixekizumab, mepolizumab, natalizumab, necitumumab, nivolumab, obiltoxaximab, obinutuzumab, ocrelizumab, ofatumumab, olaratumab, omalizumab, palivizumab, panitumumab, pembrolizumab, pertuzumab, ramucirumab, ranibizumab, reslizumab, secukinumab, siltuximab, tocilizumab, ustekinumab, vedolizumab, sarilumab, rituximab, guselkumab, inotuzumab, adalimumab, gemtuzumab, bevacizumab, benralizumab, emicizuma and trastuzumab.

The present invention also concerns a method of producing an antibody of the invention, comprising providing a host cell or a transgenic organism of the invention, culturing said a host cell or allowing said organism to grow, under conditions suitable for expression of the antibody, and optionally recovering the antibody from the host cell culture or from a sample of said organism. Optionally, the recovered antibody may be further purified. Suitable media, culture conditions and production method are well-known by the skilled person and can be easily chosen according to the host cell and the antibody to be produced.

In another aspect, the present invention further relates to a pharmaceutical composition comprising a recombinant nucleic acid molecule, an expression cassette, a vector, a viral particle, a host cell or an antibody of the invention, and a pharmaceutical acceptable excipient. The composition may comprise one or several recombinant nucleic acid molecules, one or several expression cassettes, one or several vectors, one or several viral particles, one or several host cells and/or one or several antibodies of the invention.

As used herein, the term “pharmaceutical formulation” or “pharmaceutical composition” refers to a preparation which is in such form as to permit the biological activity of the active ingredient to be effective, and which contains no additional components which are unacceptably toxic to a subject to which the formulation would be administered. Preferably, such formulations are sterile, i.e. aseptic or free from all living microorganisms and their spores.

As used herein, the term “pharmaceutically acceptable” means approved by a regulatory agency or recognized pharmacopeia such as European Pharmacopeia, for use in animals and/or humans. The term “excipient” refers to a diluent, adjuvant, carrier, or vehicle with which the therapeutic agent is administered.

Acceptable carriers, excipients, or stabilizers are nontoxic to recipients at the dosages and concentrations employed, and include, but are not limited to, buffering agents, stabilizing agents, preservatives, isotonifiers, non-ionic detergents, antioxidants and other miscellaneous additives.

As is well known in the art, pharmaceutically acceptable excipients are relatively inert substances that facilitate administration of a pharmacologically effective substance and can be supplied as liquid solutions or suspensions, as emulsions, or as solid forms suitable for dissolution or suspension in liquid prior to use. For example, an excipient can give form or consistency, or act as a diluent. Suitable excipients include but are not limited to stabilizing agents, wetting and emulsifying agents, salts for varying osmolality, encapsulating agents, pH buffering substances, and buffers. Such excipients include any pharmaceutical agent suitable for direct delivery to the eye which may be administered without undue toxicity. Pharmaceutically acceptable excipients include, but are not limited to, sorbitol, any of the various tween compounds, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington's Pharmaceutical Sciences, 15th Edition.

The form of the pharmaceutical compositions, the route of administration, the dosage and the regimen depend upon the condition to be treated, the severity of the illness, the age, weight, and sex of the patient, etc.

The doses used for the administration can be adapted as a function of various parameters, and in particular as a function of the mode of administration used, of the relevant pathology, or alternatively of the desired duration of treatment.

The pharmaceutical compositions of the invention can be formulated for a topical, oral, parenteral, intranasal, intravenous, intramuscular, subcutaneous or intraocular administration and the like.

Preferably, the pharmaceutical formulation is a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

Sterile injectable solutions may be prepared by incorporating the active compounds in the required amount in the appropriate solvent with various of the other ingredients enumerated above, as required, followed by filtered sterilization. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In addition to the compositions formulated for parenteral administration, such as intravenous or intramuscular injection, other pharmaceutically acceptable forms include, e.g. tablets or other solids for oral administration; time release capsules; and any other form currently used.

The pharmaceutical composition may further comprise one or several additional active compounds. Examples of additional active compounds include, but are not limited to, chemotherapeutic drug, antibiotics, antiparasitic agents, antifungal agents or antiviral agents.

All the embodiments of the recombinant nucleic acid molecule, the expression cassette, the vector, the viral particle, the host cell and the antibody of the invention are also contemplated in this aspect.

The present invention further relates to a recombinant nucleic acid molecule, an expression cassette, a vector, a viral particle, a host cell, an antibody or a pharmaceutical composition of the invention for use in the prevention or treatment of a disease.

The present invention relates to the use of a recombinant nucleic acid molecule, an expression cassette, a vector, a viral particle, a host cell, an antibody or a pharmaceutical composition of the invention as a medicament for the treatment of a disease. The invention also relates to the use of a recombinant nucleic acid molecule, an expression cassette, a vector, a viral particle, a host cell or an antibody of the invention for the manufacture or preparation of a medicament.

In particular, the present invention relates to a method of treating a disease in a subject, comprising administering to said subject an effective amount of a recombinant nucleic acid molecule, an expression cassette, a vector, a viral particle, a host cell, an antibody or a pharmaceutical composition of the invention.

As used herein, the term “subject” or “patient” refers to a mammal, preferably a human being.

The disease may be any disease which can be treated, prevented or alleviated through the action of an antibody, in particular an antibody of the invention. Examples of such diseases include, but are not limited to, cancer, infectious diseases, auto-immune diseases and inflammatory diseases.

All the embodiments of the recombinant nucleic acid molecule, the expression cassette, the vector, the viral particle, the host cell, the antibody and the pharmaceutical composition of the invention are also contemplated in this aspect.

The present invention further relates to a recombinant nucleic acid molecule, an expression cassette, a vector, a viral particle, a host cell, an antibody or a pharmaceutical composition of the invention, preferably an antibody of the invention, for use in a method of diagnosis or detection of a disease.

The disease to be diagnosed or detected depends on the antibody.

The method may comprise contacting the biological sample with an antibody of the invention under conditions permissive for binding of the antibody to its antigen, if present in the sample, and detecting whether a complex is formed between the antibody and its antigen. Such method may be an in vitro or in vivo method. The term “detecting” as used herein encompasses quantitative or qualitative detection.

In preferred embodiments, the antibody of the invention used in diagnostic methods is labelled. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction.

As used in this specification, the term “about” refers to a range of values±10% of the specified value, more preferably a range of values±5% of the specified value. For instance, “about 1” means from 0.9 to 1.1 when 10% is considered and from 0.95 to 1.05 when 5% is considered. As used herein, the verb “to comprise” is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

All patent and literature references cited in the present specification are hereby incorporated by reference in their entirety.

The following examples are given for purposes of illustration and not by way of limitation.

### Examples

### Material and Method

### Construction of an Anti-CD20 IgG5

Gene synthesis reaction was used to create a fully synthetic cassette encoding an anti-CD20 IgG5 (with constant sequences from the pseudo-gamma gene). Variable domains were based on the rituximab sequence. In order to obtain an antibody from the IgG5 class, we designed an original strategy by which:
full-length V_{H},DJ_{H} was linked tho the CH1, CH2 and CH3 assembled exons determined from the, supposedly non-expressed in human, pseudo-gamma gene. This sequence was then followed by a polyadenylation site.
the rituximab V_{κ}J_{κ} sequence was followed by Cₖ. This sequence was then followed by a polyadenylation site.

Finally, the constructs were cloned into the pCDNA3.1(+) vector, which contains a neomycin gene resistance for selection, at the HindIII and EcoRI sites.

### Production

CHO and 293 cells were transfected with the plasmid vectors, and selected with 0.5 to 1 mg/mL G418 complemented media the day after transfection. After 3-4 weeks, supernatants were screened for expression by an enzyme-linked immunosorbent assay, and by flow cytometry. Supernatant was eventually concentrated by centrifugation on a Vivaspin disposal with a 10-kDa cut-off.

### Characterization

For ELISA, the IgG5 was captured by polyclonal anti-human Fc-specific antibodies, and detected by HRP-conjugated anti-human IgG (H+L) antibodies (both from Sigma Aldrich). For flow cytometry, 100 000 cells expressing the CD20 membrane antigen (or negative control cells) were incubated with 50 μL of supernatant during 30 min at 2-8° C. After 3 washes with cold PBS-1% SAB, 10 μL of diluted anti-human IgG (H+L)-Dylight 650 was added to the cell suspension. Subsequently a 20-min incubation at 2-8° C., cells were washed again 3 times and finally re-suspended with 300 μl of buffer. Propidium iodide was added just before reading for exclude dead cells. 10 000 events gated on live cells were recorded on FACSCalibur cytometer.

### Results

FIG. 1a: Schematic diagrams of the IgG1 and IgG5 showing that the hinge domain of the IgG5 differs and is slightly longer than the hinge domain of IgG1.

FIG. 1b: Alignment of the 5 IgG classes protein sequences (CH1, Hinge, CH2 and CH3). Grey boxes highlight differences between IgG5 and IgG1.

FIGS. 2a and 2b: Map of the plasmid constructed for anti-CD20 IgG5 expression. (a) Construction for the VDJ sequence of the rituximab-pseudo-gamma was inserted at the XbaI and EcoRV sites into a pCDNA3.4 (+) vector. (b) Construction for the Vkappa sequence of the rituximab was inserted at the XbaI and EcoRV sites into a pCDNA3.4 (+) vector.

FIG. 3: Detection of immunoglobulins in supernatant of CHO-S cells by ELISA. Supernatant from transfected cells were collected and analyzed by ELISA. Recombinant IgG1 and IgM mAb were used as, respectively, positive and negative control. IgG5 was successfully detected in CHO-S transfected cell line supernatant.

FIG. 4: Staining of target cells with supernatant allows to show that IgG5 anti-CD20 was detected on a positive CD20 cell line (EL4-CD20), not in the negative control cells. This results shows that CHO-S cells transformed with a nucleic acid encoding IgG5 of the invention could produce the recombinant antibody (IgG5 anti-CD20) and that it was functional and could recognize its antigen.

1- A recombinant nucleic acid molecule comprising:
a sequence encoding an immunoglobulin heavy chain variable region (V_{H});
a sequence encoding the immunoglobulin heavy chain pseudo-gamma gene constant region (C_{H}) corresponding to the CH1, hinge, CH2 and CH3 protein domains encoded by the human pseudo-gamma (ψγ) gene;
a sequence encoding an immunoglobulin light chain variable region (V_{L});
a sequence encoding a light chain constant region (C_{L}).

2- The recombinant nucleic acid molecule of claim 1, wherein the sequence encoding an immunoglobulin heavy chain constant region encodes only part of the pseudo-gamma heavy chain constant domains.

3- The recombinant nucleic acid molecule of claim 1, wherein the nucleic acid molecule further comprises additional polynucleotides for directing integration of said nucleic acid molecule by homologous recombination at a precise location into a genome of a host cell.

4- The recombinant nucleic acid molecule of claim 1, wherein peptide linkers fuse antibody heavy and light chains into a single-chain antibody molecule.

5- An expression cassette comprising a recombinant nucleic acid molecule of claim 1 operably linked to one or more control sequences that direct the expression of said nucleic acid in a suitable host cell under conditions compatible with the control sequences.

6- The expression cassette of claim 5, wherein the recombinant nucleic acid molecule is operably linked to an immunoglobulin V_{H} promoter.

7- A vector comprising an expression cassette of claim 5.

8- The vector of claim 7, wherein said vector is a viral vector.

9- The vector of claim 7, wherein said vector is an adeno-associated viral (AAV) vector.

10- A viral particle comprising a vector of claim 7.

11- An isolated cell comprising a recombinant nucleic acid molecule of claim 1.

12- The cell of claim 11, wherein said cell is a mouse embryonic stem cell.

13- The cell of claim 11, wherein said cell is B cell.

14- A non-human animal transgenic organism, comprising at least one cell as defined in claim 11.

15- An antibody comprising:
an immunoglobulin heavy chain variable region (V_{H});
an immunoglobulin light chain variable region (V_{L});
an immunoglobulin light chain constant region (C_{L});
an immunoglobulin heavy chain constant region (C_{H}) including all or part of the peptides encoded by the human immunoglobulin pseudo-gamma gene.

16- A method of producing an antibody comprising:
an immunoglobulin heavy chain variable region (V_{H});
an immunoglobulin light chain variable region (V_{L});
an immunoglobulin light chain constant region (C_{L});
an immunoglobulin heavy chain constant region (C_{H}) including all or part of the peptides encoded by the human immunoglobulin pseudo-gamma gene, the method comprising:
providing a cell as defined in claim 11, said cell expressing said antibody, culturing said cell, and recovering said antibody from the cell culture.

17- A pharmaceutical composition comprising a recombinant nucleic acid molecule of claim 1, and a pharmaceutically acceptable excipient.

18- The vector of claim 8, wherein the vector is a lentiviral vector.

19- The vector of claim 9, wherein the vector is an AAV6 vector.

20- The cell of claim 13, wherein said cell human B cell.


SEQUENCE LISTING (5 sequences)

SEQ ID NO: 1 - protein, 330 aa, Homo sapiens
  Gamma 1
RSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 326 aa, Homo sapiens
  Gamma 2
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 3 - protein, 377 aa, Homo sapiens
  Gamma 3
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 4 - protein, 327 aa, Homo sapiens
  Gamma 4
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 331 aa, Homo sapiens
  Gamma 5
ASTKGPSVFPLVPSSRSVSEGTAALGCLVKDYFPEPVTVSWNSGALTRSVHTFPAVLQSSGLYSLSSVVTVPSSSLGTQTYTCNVDHKPSNTKVDKTVEPKTPCCDTTHTCPPCATTEPLGGPSVFLFPPKPKDTLMISRTPEVTCVVVDVSHEDPEVKFNWYVDGVEVHNAKTKPWEEQYNSTYHVVSVLTVVHQNWLNGREYKCKVSNKGLPAPIEKTISKTKGQPREPQVYTLPPSQKMTKNQVTLTCLVKGFYPSDITVEWESNGQPENNYKTTPPMLDSNGSFFLYSKLTVDKSRWQQGNVFSCSVMHEGLHNHYTQKSLSLSPGK